(19) United States
(12) Patent Application Publication
(10) Pub. No.: US 2023/0001026 A1
(43) Pub. Date: Jan. 5, 2023

(54) PHARMACEUTICAL PACKAGING UNITS AND METHODS FOR CONCOMITANT ADMINISTRATION OF RADIOTRACERS

(71) Applicant: LikeMinds, Inc., Boston, MA (US)

(72) Inventors: Reinhard Koenig, Boston, MA (US); William Dawes, Boston, MA (US)

(21) Appl. No.: 17/778,754

(22) PCT Filed: Nov. 20, 2020
(86) PCT No.: PCT/US20/61597

§ 371 Date: May 20, 2022

Related U.S. Application Data
(60) Provisional application No. 62/938,908, filed on Nov. 21, 2019.

(51) Int. Cl. A61K 51/04

(52) U.S. Cl. CPC A61K 51/0448

(57) ABSTRACT

This disclosure relates to pharmaceutical packaging units comprising different radiotracers and methods of using the same.

### RELATED APPLICATIONS

This application claims priority to U.S. Provisional Application Nos. 62/938,908, filed Nov. 21, 2019, which is incorporated herein by reference in its entirety.

### FIELD OF THE INVENTION

The present invention is in the field of human and veterinary medicine. More specifically, the invention relates to pharmaceutical packaging units and methods of diagnosis of dopaminergic and non-dopaminergic disorders.

### BACKGROUND OF THE INVENTION

Movement disorders are common among adults and have significant personal, financial, and societal impact. Varying degrees of tremor are often manifested, with severe tremor causing difficulty or the inability to perform routine activities. Many of these disorders are progressive and may proceed rapidly (Zesiewicz et al. (2005) Neurol. 64(12): 2008-20; Stone (1991) Pharmacol. Biochem. Behav. 39(2): 345-9; Stone (1995) J. Neurol. Sci. 132(2):129-32.

Movement disorders are usually or initially diagnosed clinically and may be based on questionnaires, non-motor symptoms such as REM sleep disorders, constipation, and observation of voluntary and involuntary movement. For example, in the case of the most common movement disorder, non-parkinsonian tremor (also called Essential tremor, benign tremor, familial tremor, or idiopathic tremor), diagnosis is usually established by observing an action tremor (i.e., a tremor which intensifies when one tries to use the affected muscles of hands, arms, and/or fingers) or postural tremor (i.e., present with sustained muscle tone), rather than the tremor exhibited at rest (resting tremor), which is manifested in Parkinson's Disease. When a limb is at rest, no tremor is observed, but moving or extending the limb results in a shaking. This is in contrast to resting tremor which is manifested in Parkinson's disease.

Unfortunately, clinical diagnoses of movement disorders are at best speculative many disorders display similar physical symptoms which can also be confused with other tremor disorders.

Definitive diagnosis of a movement disorder is possible only during autopsy when histological examination of regions of the brain, e.g., the basal ganglia, particularly the substantia nigra (SN), is performed. This is because the dopaminergic pathway from the SN to the striatum is known to play an integral part in motor function and is involved in many movement disorders (“dopaminergic” disorders). However, not all tremor disorders involve this pathway. For example, there are movement disorders that, in the earlier stages, may manifest as Parkinson's Disease clinically, but there is no evidence of dopamine deficiency in the striatum, thus suggesting the movement disorder etiology is a type of non-parkinsonian syndrome (such as essential tremor, a “non-dopaminergic movement disorder) which requires a different clinical treatment protocol. Therefore, without information to rule out the involvement of the SN to striatum dopamine pathway, clinical diagnoses of movement disorders are at best speculative.

More recently, the SN to striatum pathway of a live patient has become assessible by viewing images of the striatum obtained by various noninvasive imaging techniques. Such non-invasive methods use single photon emission computed tomography (SPECT) assessment measuring the uptake/binding of a radiolabeled imagining agent that binds to dopamine transporters (DAT) in the striatum. Currently, the only SPECT radiopharmaceutical approved for use in the United States, the European Union, and Canada is DaTSCAN® (N-ω-fluoropropyl-2β-carbomethoxy-3β-(4-iodophenyl) nortropane), which is radiolabeled with ¹²³I. After injection of DaTSCAN, the patient must wait 3 hr for its uptake into systemic circulation, and transition through the blood brain barrier into the parenchyma of the brain tissue until it reaches stable binding to DAT. At that point, radioactivity (counts) can be collected and quantified, and a visual image can be acquired via SPECT. The visual image is compiled for reading by experts who then determine if there is evidence of DAT density loss in the striatum. In the case of a patient with Parkinson's disease, an asymmetrical pattern of binding in the striatum is observed in the image, whereas in the non-parkinsonian tremor patient, the pattern is visible as bilateral and symmetrical (i.e., “normal) (Lewis et al. (2012) Practitioner 1748: 21-24). This information is used by treating physicians in determining diagnosis and subsequent treatment.

Unfortunately, DaTSCAN is also highly selective for serotonin (SERT) transporters (for every two to three DAT sites DaTSCAN binds to one SERT site) in the lungs and other tissues (DaTscan™ Ioflupane ¹²³I Injection (package insert), Arlington Heights, Ill.: GE Healthcare, Medi-Physics, Inc.; 2015). Due to DaTscan's selectivity for SERT and the slow transition time of the compound to the neurons in the SN, imaging cannot take place until 3 to 6 hr after injection, at which time its binding to striatal DAT becomes stable. The patient is then placed in the SPECT camera until 1.5 million counts are obtained (approximately 45 min). The binding of DaTSCAN to transporters other than DAT leaves only about 7% of DaTSCAN available for DAT transporter binding in the brain tissue (DaTscan™ Ioflupane ¹²³I Injection (package insert), ibid.). Thus, a significant disadvantage of the only currently approved imaging agent used with this technology is that an undetermined but significant amount of DaTSCAN is being bound and captured by serotonin transporters throughout the body and, therefore, is not available for specific binding to neuronal DAT in the striatum.

DAT is also found outside the brain in nephrons, kidney, pancreas, lungs, and the cardio-pulmonary system including many blood vessels outside the CNS. Dysfunctions of DAT outside the brain are known.

There is thus a need for a more accurate and rapid way to diagnose movement disorders which can distinguish non-dopaminergic disorders from dopaminergic disorders, so that efficacious therapeutic treatment specific for the disorder can be administered more quickly. There is additionally a need for ways to diagnose DAT-related disorders outside of the brain.

### SUMMARY OF THE INVENTION

It has been discovered that the imaging agent [¹²³I]-E-2β-carbomethoxy-3β-(4-fluorophenyl)-N-(3-iodo-E-allyl) nortropane (DaT2020) has a binding selectivity of at least 28-fold for DAT than it does for SERT. This greater binding selectivity enables an increase in the availability of tracer which can penetrate more deeply into brain tissues, and can bind more quickly to DAT than other known radiolabeled tropane imaging agents.

These discoveries have been exploited to provide the present pharmaceutical packaging units and methods useful to quickly and accurately determine if a patient afflicted with a movement disorder has a malfunction of the dopaminergic system or has a non-dopaminergic movement disorder or other disorder that is difficult to diagnose through clinical observations alone.

In one aspect, the disclosure provides a pharmaceutical packaging unit comprising a first radiotracer and a second radiotracer, wherein the first radiotracer and the second radiotracer have different binding affinities for the dopamine transporter (DAT).

In some embodiments, the binding affinity of the first radiotracer for DAT is between about 2fold- to about 50-fold greater than the binding affinity of the second radiotracer for DAT. In some embodiments, the binding affinity of the first radiotracer for DAT is between about 2-fold to about 5-fold greater than the binding affinity of the second radiotracer for DAT. In some embodiments, the binding affinity of the first radiotracer for DAT is between about 5- to about 10-fold greater than the binding affinity of the second radiotracer for DAT. In some embodiments, the binding affinity of the first radiotracer for DAT is between about 10- to about 20-fold greater than the binding affinity of the second radiotracer for DAT. In some embodiments, the binding affinity of the first radiotracer for DAT is between about 20- to about 30-fold greater than the binding affinity of the second radiotracer for DAT. In some embodiments, the binding affinity of the first radiotracer for DAT is between about 30- to about 40-fold greater than the binding affinity of the second radiotracer for DAT. In some embodiments, the binding affinity of the first radiotracer for DAT is between about 40- to about 50-fold greater than the binding affinity of the second radiotracer for DAT.

In another aspect, the disclosure provides a pharmaceutical packaging unit comprising a first radiotracer and a second radiotracer, wherein the first radiotracer and the second radiotracer have different pharmacokinetics.

In some embodiments, the pharmacokinetics of the first radiotracer is between about 2-fold to about 500-fold greater than the pharmacokinetics of the second radiotracer. In some embodiments, the pharmacokinetics of the first radiotracer is between about 2- to about 50-fold greater than the pharmacokinetics of the second radiotracer. In some embodiments, the pharmacokinetics of the first radiotracer is between about 50- to about 100-fold greater than the pharmacokinetics of the second radiotracer. In some embodiments, the pharmacokinetics of the first radiotracer is between about 100- to about 200-fold greater than the pharmacokinetics of the second radiotracer. In some embodiments, the pharmacokinetics of the first radiotracer is between about 200- to about 300-fold greater than the pharmacokinetics of the second radiotracer. In some embodiments, the pharmacokinetics of the first radiotracer is between about 300- to about 400-fold greater than the pharmacokinetics of the second radiotracer. In some embodiments, the pharmacokinetics of the first radiotracer is between about 400- to about 500-fold greater than the pharmacokinetics of the second radiotracer.

In another aspect, the disclosure provides a pharmaceutical packaging unit comprising a first radiotracer and a second radiotracer, wherein the first radiotracer and the second radiotracer have different binding affinities for the dopamine transporter (DAT) and different pharmacokinetics.

In some embodiments, the binding affinity of the first radiotracer for DAT is between about 2- to about 50-fold greater than the binding affinity of the second radiotracer for DAT and the pharmacokinetics of the first radiotracer is about 2- to about 500-fold greater than the pharmacokinetics of the second radiotracer. In some embodiments, the binding affinity of the first radiotracer for DAT is between about 2- to about 5-fold greater than the binding affinity of the second radiotracer for DAT and the pharmacokinetics of the first radiotracer is between about 2- to about 50-fold greater than the pharmacokinetics of the second radiotracer. In some embodiments, the binding affinity of the first radiotracer for DAT is between about 5- to about 500-fold greater than the binding affinity of the second radiotracer for DAT and the pharmacokinetics of the first radiotracer is between about 50- to about 100-fold greater than the pharmacokinetics of the second radiotracer. In some embodiments, the binding affinity of the first radiotracer for DAT is between about 10- to about 20-fold greater than the binding affinity of the second radiotracer for DAT and the pharmacokinetics of the first radiotracer is between about 100- to about 200-fold greater than the pharmacokinetics of the second radiotracer. In some embodiments, the binding affinity of the first radiotracer for DAT is between about 20- to about 30-fold greater than the binding affinity of the second radiotracer for DAT and the pharmacokinetics of the first radiotracer is between about 200- to about 300-fold greater than the pharmacokinetics of the second radiotracer. In some embodiments, the binding affinity of the first radiotracer for DAT is between about 30- to about 40-fold greater than the binding affinity of the second radiotracer for DAT and the pharmacokinetics of the first radiotracer is between about 300- to about 400-fold greater than the pharmacokinetics of the second radiotracer. In some embodiments, the binding affinity of the first radiotracer for DAT is between about 40- to about 50-fold greater than the binding affinity of the second radiotracer for DAT and the pharmacokinetics of the first radiotracer is between about 400- to about 500-fold greater than the pharmacokinetics of the second radiotracer.

In some embodiments of the pharmaceutical packaging units disclosed herein, the first radiotracer comprises or is [¹²³I E-2β-carbomethoxy-3β-(4-fluorophenyl)-N-(3-iodo-E-allyl) nortropane (DaT2020). In some embodiments of the pharmaceutical packaging units disclosed herein, the second radiotracer comprises or is N-ω-fluoropropyl-2β-carbomethoxy-3β-(4-iodophenyl) nortropane (DaTSCAN).

In another aspect, the disclosure provides a method of determining if a subject not manifesting a clinical symptom of a dopaminergic disorder is afflicted with the dopaminergic disorder, comprising: administering a first radiotracer; waiting for a first time interval; administering a second radiotracer; waiting for a second time interval; acquiring counts from the first radiotracer and the second radiotracer, bound to DAT in a region of interest (ROI) of the body of the subject; measuring a number, density, and/or pattern of counts acquired; and comparing the number, density, and/or pattern of counts acquired from the ROI of the subject with the number, density, and/or pattern of counts obtained from an unafflicted, age-matched control subject, the patient being afflicted with a dopaminergic movement disorder if the number, density and/or pattern of counts detected in the ROI is reduced relative to the counts, density, and/or pattern of counts obtained from the ROI the unafflicted, age-match control subject, wherein the first radiotracer and the second radiotracer have different binding affinities for the dopamine transporter (DAT) and different pharmacokinetics.

In another aspect, the disclosure provides a method of determining if a subject not manifesting a clinical symptom of a dopaminergic disorder is afflicted with the dopaminergic disorder, comprising: administering a second radiotracer; waiting for a first time interval; administering a first radiotracer; waiting for a second time interval; acquiring counts from the first radiotracer and the second radiotracer, bound to DAT in a region of interest (ROI) of the body of the subject; measuring a number, density, and/or pattern of counts acquired; and comparing the number, density, and/or pattern of counts acquired from the ROI of the subject with the number, density, and/or pattern of counts obtained from an unafflicted, age-matched control subject, the patient being afflicted with a dopaminergic movement disorder if the number, density and/or pattern of counts detected in the ROI is reduced relative to the counts, density, and/or pattern of counts obtained from the ROI the unafflicted, age-match control subject, wherein the first radiotracer and the second radiotracer have different binding affinities for the dopamine transporter (DAT) and different pharmacokinetics.

In some embodiments of the methods disclosed herein, the first time interval is between about 5 minutes to about 6 hours. In particular embodiments, the first time interval is from about 5 minutes to about 30 minutes, between about 0.5 hours to about 1 hour, between about 1 hours to about 2 hours, between about 2 hours to about 3 hours, between about 3 hours to about 4 hours, between about 4 hours to about 5 hours, or between about 5 hours to about 6 hours. In some embodiments of the methods disclosed herein, the second time interval is between about 5 minutes to about 30 minutes, between about 0.5 hours to about 1 hour, between about 1 hours to about 2 hours, between about 2 hours to about 3 hours, between about 3 hours to about 4 hours, between about 4 hours to about 5 hours, or between about 5 hours to about 6 hours.

In some embodiments of the methods disclosed herein, the binding affinity of the first radiotracer for DAT is between about 2-fold to about 50-fold greater than the binding affinity of the second radiotracer for DAT. In some embodiments of the methods disclosed herein, the pharmacokinetics of the first radiotracer is between about 2-fold to about 500-fold greater than the pharmacokinetics of the second radiotracer.

In some embodiments of the methods disclosed herein, the first radiotracer comprises or is [¹²³I E-2β-carbomethoxy-3β-(4-fluorophenyl)-N-(3-iodo-E-allyl) nortropane (DaT2020). In some embodiments of the methods disclosed herein, the second radiotracer comprises or is N-ω-fluoropropyl-2β-carbomethoxy-3β-(4-iodophenyl) nortropane (DaTSCAN).

### DETAILED DESCRIPTION

The disclosures of any patents, patent applications, and publications referred to herein are hereby incorporated by reference in their entireties into this application in order to more fully describe the state of the art as known to those skilled therein as of the date of the invention described and claimed herein. The instant disclosure will govern in the instance that there is any inconsistency between the patents, patent applications, and publications and this disclosure.

Unless defined otherwise, all technical and scientific terms used herein have the same meaning as commonly understood by one of ordinary skill in the art to which this disclosure belongs. The initial definition provided for a group or term herein applies to that group or term throughout the present specification individually or as part of another group, unless otherwise indicated.

As used herein, the term “DAT” or “DAT receptor” refers to a dopamine transporter receptor.

As used herein, the term “DaT2020” refers to E-2β-carbomethoxy-3β-(4-fluorophenyl)-N-(3-iodo-E-allyl) nortropane.

As used herein, the term “agent” or “tracer” or refers to radiolabeled DaT2020 and radiolabeled derivatives thereof.

As used herein, the term “radiotracer” refers to any radiolabeled compound, and derivatives thereof, that acts as a functional tracer for DAT. Examples of a radiotracer include, but are not limited to, DaT2020 and DaTSCAN.

As used herein, the term “tropane” refers to DaT2020 and its derivatives.

As used herein, the phrase “radiolabeled tropane” or “radiolabeled DaT2020” refer to radiolabeled DaT2020, and derivatives thereof, labeled with ¹²³I, ¹²⁴I, ¹²⁵I, 18F, ⁹⁹mTc, ¹¹C, or ¹¹⁷mSn. The term “Altropane” refers specifically to [¹²³I E-2β-carbomethoxy-3β-(4-fluorophenyl)-N-(3-iodo-E-allyl) nortropane.

As used herein, the phrase “differential diagnosis” refers to a process of differentiating between two or more conditions that share similar clinical signs and/or symptoms.

As used herein, the term “administer” or “administering” refers to those methods used to introduce a substance into the system of a subject. Such methods include parenteral administration, e.g., intravenously. The intravenous route represents the most efficient way to deliver an in vivo imaging agent throughout the body of the subject and therefore into contact with one or more defined biological markers expressed in said subject. Furthermore, intravenous administration does not represent a substantial physical intervention or a substantial health risk. The in vivo imaging agent may be administered as a pharmaceutical composition.

As used herein the phrase, “pharmaceutical composition” or “pharmaceutical packaging unit” refers to a biologically active compound, i.e., radiolabeled tropane, together with a pharmaceutically acceptable carrier in a form suitable for mammalian administration.

As used herein, the phrase “pharmaceutically acceptable carrier” is a fluid, especially a liquid, in which the radiolabeled tropane is suspended or dissolved, such that the composition is physiologically tolerable, i.e. can be administered to the mammalian body without toxicity or undue discomfort. The pharmaceutically acceptable carrier is suitably an injectable carrier liquid such as sterile, pyrogen-free water for injection; an aqueous solution such as saline (which may advantageously be balanced so that the final product for injection is either isotonic or not hypotonic); an aqueous solution of one or more tonicity-adjusting substances (e.g. salts of plasma cations with biocompatible counterions), sugars (e.g. glucose or sucrose), sugar alcohols (e.g. sorbitol or mannitol), glycols (e.g. glycerol), or other non-ionic polyol materials (e.g. polyethyleneglycols, propylene glycols and the like). The pharmaceutically acceptable carrier may also comprise biocompatible organic solvents such as ethanol. Such organic solvents are useful to solubilize more lipophilic compounds or formulations. Preferably the pharmaceutically acceptable carrier is pyrogen-free water for injection, isotonic saline or an aqueous ethanol solution. The pH of the pharmaceutically acceptable carrier for intravenous injection is suitably in the range 4.0 to 10.5.

As used herein, the phrase “in a form suitable for mammalian administration” refers to a composition which is sterile, pyrogen-free, lacks compounds which produce toxic or adverse effects, and is formulated at a biocompatible pH (approximately pH 4.0 to 10.5). Such compositions lack particulates which could risk causing emboli in vivo, and are formulated so that precipitation does not occur on contact with biological fluids (e.g. blood). Such compositions also contain only biologically compatible excipients, and are preferably isotonic.

### Pharmaceutical Packaging Units Comprising Radiotracers

The present disclosure provides, at least in part, pharmaceutical packaging units comprising at least the radiolabeled imaging agent, DaT2020, or derivatives thereof, to quickly distinguish dopaminergic disorders from non-dopaminergic disorders, and to image dopamine transporters (DATs) in different regions of the brain and body involved in such disorders. DaT2020 is highly advantageous as it is more selective for, and binds more quickly to, DAT than other commercially available imaging agents. The present disclosure provides also, at least in part, pharmaceutical packaging units comprising at least DaT2020, or derivatives thereof, and another different radiolabeled imaging agent that has an affinity for DAT different from the affinity of DaT2020 for DAT.

The imaging agent used in the present pharmaceutical packaging unit is the tropane, DaT2020, and derivatives thereof (collectively, “DaT2020”). These imaging agents have a higher binding selectivity for DAT over SERT (28:1 or 28-fold), bind more DAT quickly, and penetrate brain tissue and other regions of interest more deeply than other known DAT tropane tracers currently used (e.g., DaTSCAN) (DaTSCAN Ioflupane ¹²³I Injection [package insert] (2015) Arlington Heights, Ill.: GE Healthcare, Medi-Physics, Inc.).

Dat2020 travels in the blood stream and into the brain more quickly and binds more specifically to DAT than DaTSCAN. Because of the favorable pharmacokinetic profile of DaT2020 stable binding occurs 15 min after administration allowing scanning to begin and resulting in sufficient data for an image to be collected over 30 min. In contrast, the scanning procedure with DaTSCAN cannot begin until at least 180 min after administration and usually the scan lasts about 45 min.

Non-limiting examples of DaT2020 derivatives include 2β carbomethoxy-3β-(4-iodophenyl) tropane (beta-CIT); 2β-carbomethoxy-3β-(4-iodophenyl)-N-(3-fluoropropyl) nortropane (FP-CIT); and TRODAT-1. These derivatives are described in U.S. Pat. Nos. 5,493,026, 8,084,018, 8,574,545, 8,986,653, and PCT International Application No. PCT/US2015/037340. DaT2020 can be commercially obtained (from LikeMinds, Inc.) or can be synthesized, e.g., according to U.S. Pat. Nos. 8,986,653 and 8,574,545).

Radiolabeled DaT2020 and its radiolabeled derivatives may be generated by the user through a radiolabeling procedure. For example, to prepare DaT2020, one may allow a reaction between a haloallyl Sn precursor (pre-DaT2020) and a radionuclide under oxidative conditions. Other standard methods of radiolabeling can be used as well. For radiolabeling, DaT2020 in lyophilized form is useful, however, it can also be in aqueous form.

The radiolabel bound to the tropane is one that is detectable via SPECT (as shown above), including ¹²³I, ¹²⁴I, ¹²⁵I, ⁹⁹mTc, or ^{117m}Sn or PET (as shown above), including ¹⁸F or ¹¹C. The location of the radioisotope on the agent can be varied. For example, the isotope can be located at any position on pre-DaT2020 or a derivative thereof and can be directly linked or indirectly linked via a linker (see, U.S. Pat. No. 8,574,545). One suitable position is the free terminus of the haloallyl moiety.

Non-limiting examples of useful SPECT-readable tracers for DAT detection according to the disclosure include [¹²³I]-E-2β-carbomethoxy-3β-(4-fluorophenyl)-N-(3-iodo-E-allyl) nortropane (DaT2020), [¹²³I]-2β carbomethoxy-3β-(4-iodophenyl)tropane ([¹²³I]-beta-CIT); [¹²³I]-2β-carbomethoxy-3β-(4-iodophenyl)-N-(3-fluoropropyl)nortropane ([¹²³I]-FP-CIT); [¹²³I]-altropane; and [⁹⁹mTc]-TRODAT-1. Among these, [¹²³I]-FP-CIT (DaTSCAN) achieves stable binding 3 hr post-injection and remains stable for 3 hr, has a half-life of 13.2 hr, emits gamma rays with an energy of 159 keV, and is FDA approved. These are also described in U.S. Pat. Nos. 5,493,026, 8,084,018, 8,574,545, 8,986,653, and PCT International Application No. PCT/US2015/037340. The movement, and binding, of radiolabeled DaT2020 to DAT can also be performed using other methods of monitoring radioactive compounds used, e.g., by radioactivity sensors located on the head of the patient adjacent to the region of interest (ROI), or on other regions of the body where DATs are located and are being monitored for DaT2020 binding.

The use of the pharmaceutical packaging units disclosed herein can distinguish non-dopaminergic conditions, such as, but not limited to, non-parkinsonian or essential tremor and non-Alzheimer dementia, as well as multiple sclerosis, chronic kidney disease, stroke, traumatic brain injury, drug or alcohol use, hypoglycemia, lack of sleep, lack of vitamins, increased stress, magnesium and/or thiamine deficiencies, liver failure, mercury poisoning, and drug or alcohol addiction or withdrawal, from a dopaminergic disorder displaying similar clinical manifestations. Such dopaminergic disorders include, but are not limited to, parkinsonian syndromes including idiopathic Parkinson's disease, progressive supranuclear palsy (PSP), multiple system atrophy (MSA), corticobasal degeneration (CBD), and vascular parkinsonism (VaP), among other rarer causes of parkinsonism), and Lewy body dementia, ADHD, clinical depression, anxiety, sleep disorders, obesity, sexual dysfunction, schizophrenia, pheochromocytoma, binge eating disorder, and diabetes and other disorders resulting from DAT dysfunction outside of the CNS.

The radiolabeled DaT2020 can be formulated into pharmaceutical packaging units for intravenous (IV) systemic or direct local administration in a carrier or physiological buffer that does not inhibit its binding to DAT. Such buffers include, but are not limited to, ethanol (solvent), sodium hydroxide and acetic acid (pH adjustment), sodium chloride injection (isotonic vehicle). The formulation to be administered as a bolus injection has been shown in sponsored clinical trials to be safe containing a dose of up to about 8 mCi to 10 mCi of radiolabeled DaT2020 and delivered in a total volume of about 2.5 mL to about 5 mL.

The radioactivity of the dose of radiolabeled DaT2020 or of its radiolabeled derivatives, can be determined by one with skill in the art, e.g. by a nuclear medicine imaging technician. In addition, the total radioactivity of the actual administered dose is of relevance and not the volume administered to achieve this dose. The exact radioactive dose of the tracer administered is determined by calculating the difference between the radioactivity in the syringe and delivery system before and after injection. After the dose is delivered, the syringe is filled with a volume of saline equal to the administered dose volume. The syringe content is recounted under the same conditions as used to determine the dose; separately. Useful dose ranges from about 0.25 mCi to about 1 mCi, from about 1 mCi to about 10 mCi, from about 2 mCi to about 10 mCi, from about 3 mCi to about 7 mCi, from about 5 mCi to about 8 mCi, or about 2 mCi, about 3 mCi, about 4 mCi, about 5 mCi, about 6 mCi, about 7 mCi, about 8 mCi, about 9 mCi, or about 10 mCi. Dose can alternatively be described as effective dose (for 5 mCi of radiolabeled DaT2020) of approximately 4.3 mSv. Injected dose values outside the above stated range, i.e., values lower than about 0.25 mCi or higher than about 10 mCi are considered as potential sources of variation.

DaT2020 or derivatives formulated as described above for IV delivery are administered in a single (bolus) dose via a syringe (e.g., a peripheral 18 to 22-gauge venous catheter inserted for the radiopharmaceutical/tracer infusion). After injection, the tracer passes through the blood-brain barrier and quickly binds to DAT if it is available. Other methods of administration can also be utilized, for example, the direct injection of suitable amounts into the brain arteries via a syringe or catheters following established procedures in neuroradiology, or arteries contiguous to the striatum or other ROI.

In common medical practice, a radiotracer for brain imaging and other imaging applications is administered as one instance of a radiotracer that fulfills a specific purpose. For example, a radiotracer like ioflupane (DaTSCAN) would be administered in patients needing verification of dopaminergic deficit to confirm or rule out a diagnosis of essential tremor or Parkinson's Disease. The kinetics and receptor binding profile (SERT:DAT) of ioflupane would then achieve a readable SPECT image of the Region of Interest (ROI) in the brain after a certain time.

For DaT2020, the high specificity for DAT receptors vs. SERT (serotonin) receptors would yield a different kinetic and receptor binding which will ultimately result in a faster and more specific SPECT image for the ROI.

Because the pharmacokinetics and the low specificity for DAT of ioflupane vs. the high specificity of DaT2020 for DAT distinguishes both compounds not only in their kinetics and SPECT image availability after i.v. injection time (45-60 post i.v. injection for DaT2020, 180-240 minutes for ioflupane post i.v. injection), but also in their binding duration to dopaminergic neurons in a ROI (for example the substantia nigra of the putamen, a basal ganglion of the brain responsible for coordination of motor neurons). DaT2020 is attached and then washed out quickly from DAT receptors located in dopaminergic neurons (within 60 minutes post i.v. injection), ioflupane is attached to and then washed out more slowly from DAT receptors located in dopaminergic neurons (within 180-240 or more minutes post i.v. injection).

Utilizing differences in kinetics and receptor binding profile, two suitable radiotracers can be administered concomitantly or in close timely proximity, each at reduced doses compared to single dose administration of the single instance of each tracer, to visualize the following characteristics of a ROI. Using the example of Parkinson's Disease (PD), the following functional and anatomical parameters of the ROI can be imaged: Timing of visualization of dopaminergic deficits, distinguishing between different underlying pathogenesis of PD. Timing of decay visualization of filling defects or lack of filling defects. Anatomical structures of a ROI visualized differentially by high vs. low receptor binding. Differential binding may indicate functional states that are not yet well understood by may be important for the diagnosis, treatment, and prognosis for a chronic degenerative brain disease such as PD.

The present disclosure provides a pharmaceutical packaging unit comprising a first radiotracer and a second radiotracer, wherein the first radiotracer and the second radiotracer have different binding affinities for the dopamine transporter (DAT).

In some examples, the binding affinity of the first radiotracer for DAT is between about 2- to about 50-fold greater than the binding affinity of the second radiotracer for DAT. In some examples, the binding affinity of the first radiotracer for DAT is between about 2- to about 5-fold greater than the binding affinity of the second radiotracer for DAT. In some examples, the binding affinity of the first radiotracer for DAT is between about 5- to about 10-fold greater than the binding affinity of the second radiotracer for DAT. In some examples, the binding affinity of the first radiotracer for DAT is between about 10- to about 20-fold greater than the binding affinity of the second radiotracer for DAT. In some examples, the binding affinity of the first radiotracer for DAT is between about 20- to about 30-fold greater than the binding affinity of the second radiotracer for DAT. In some examples, the binding affinity of the first radiotracer for DAT is between about 30- to about 40-fold greater than the binding affinity of the second radiotracer for DAT. In some examples, the binding affinity of the first radiotracer for DAT is between about 40- to about 50-fold greater than the binding affinity of the second radiotracer for DAT.

The present disclosure provides a pharmaceutical packaging unit comprising a first radiotracer and a second radiotracer, wherein the first radiotracer and the second radiotracer have different pharmacokinetics.

In some examples, the pharmacokinetics of the first radiotracer is between about 2- to about 500-fold greater than the pharmacokinetics of the second radiotracer. In some examples, the pharmacokinetics of the first radiotracer is between about 2- to about 50-fold greater than the pharmacokinetics of the second radiotracer. In some examples, the pharmacokinetics of the first radiotracer is between about 50- to about 100-fold greater than the pharmacokinetics of the second radiotracer. In some examples, the pharmacokinetics of the first radiotracer is between about 100- to about 200-fold greater than the pharmacokinetics of the second radiotracer. In some examples, the pharmacokinetics of the first radiotracer is between about 200- to about 300-fold greater than the pharmacokinetics of the second radiotracer. In some examples, the pharmacokinetics of the first radiotracer is between about 300- to about 400-fold greater than the pharmacokinetics of the second radiotracer. In some examples, the pharmacokinetics of the first radiotracer is between about 400- to about 500-fold greater than the pharmacokinetics of the second radiotracer.

The present disclosure provides a pharmaceutical packaging unit comprising a first radiotracer and a second radiotracer, wherein the first radiotracer and the second radiotracer have different binding affinities for the dopamine transporter (DAT) and different pharmacokinetics.

In some examples, the binding affinity of the first radiotracer for DAT is between about 2- to about 50-fold greater than the binding affinity of the second radiotracer for DAT and the pharmacokinetics of the first radiotracer is about 2- to about 500-fold greater than the pharmacokinetics of the second radiotracer. In some examples, the binding affinity of the first radiotracer for DAT is between about 2- to about 5-fold greater than the binding affinity of the second radiotracer for DAT and the pharmacokinetics of the first radiotracer is between about 2- to about 50-fold greater than the pharmacokinetics of the second radiotracer. In some examples, the binding affinity of the first radiotracer for DAT is between about 5- to about 10-fold greater than the binding affinity of the second radiotracer for DAT and the pharmacokinetics of the first radiotracer is between about 50- to about 100-fold greater than the pharmacokinetics of the second radiotracer. In some examples, the binding affinity of the first radiotracer for DAT is between about 10- to about 20-fold greater than the binding affinity of the second radiotracer for DAT and the pharmacokinetics of the first radiotracer is between about 100- to about 200-fold greater than the pharmacokinetics of the second radiotracer. In some examples, the binding affinity of the first radiotracer for DAT is between about 20- to about 30-fold greater than the binding affinity of the second radiotracer for DAT and the pharmacokinetics of the first radiotracer is between about 200- to about 300-fold greater than the pharmacokinetics of the second radiotracer. In some examples, the binding affinity of the first radiotracer for DAT is between about 30- to about 40-fold greater than the binding affinity of the second radiotracer for DAT and the pharmacokinetics of the first radiotracer is between about 300- to about 400-fold greater than the pharmacokinetics of the second radiotracer. In some examples, the binding affinity of the first radiotracer for DAT is between about 40- to about 50-fold greater than the binding affinity of the second radiotracer for DAT and the pharmacokinetics of the first radiotracer is between about 400- to about 500-fold greater than the pharmacokinetics of the second radiotracer.

In some examples of the pharmaceutical packaging units disclosed herein, the first radiotracer is [¹²³I E-2β-carbomethoxy-3β-(4-fluorophenyl)-N-(3-iodo-E-allyl) nortropane (DaT2020). In some examples of the pharmaceutical packaging units disclosed herein, the second radiotracer is N-ω-fluoropropyl-2β-carbomethoxy-3β-(4-iodophenyl) nortropane (DaTSCAN).

In some examples of the pharmaceutical packaging units disclosed herein, the pharmaceutical packaging units can be used in diagnosing the etiology of an underlying disease. For example, vascular, degenerative, idiopathic and other causes can be determined by visualization of the effects of the underlying properties of the first radiotracer and the second radiotracer.

### Methods Using Radiotracers

The present disclosure provides a method of determining if a subject not manifesting a clinical symptom of a dopaminergic disorder is afflicted with the dopaminergic disorder, comprising: administering radiolabeled DaT2020, or a radiolabeled derivative thereof, to the subject; and acquiring counts from the radiolabeled DaT2020, or derivative thereof, bound to DAT in a region of interest (ROI) of the body of the subject, initiation of the acquisition of counts beginning at about 15 min after administration; measuring a number, density, and/or pattern of counts acquired; and comparing the number, density, and/or pattern of counts acquired from the ROI of the subject with the number, density, and/or pattern of counts obtained from an unafflicted, age-matched control subject, the patient being afflicted with a dopaminergic movement disorder if the number, density and/or pattern of counts detected in the ROI is reduced relative to the counts, density, and/or pattern of counts obtained from the ROI the unafflicted, age-match control subject.

In some examples, the method further comprises repeating the method at a set period of time after the method is first performed. In some examples, the counts obtained from the unafflicted, age-matched control subject is an average of counts, density, and/or patterns obtained from a plurality of unafflicted, age-matched control subjects. In some examples, the ROI is the striatum, putamen, kidney, pancreas, or a part of the cardio-vascular system of the subject. In some examples, DaT2020, or a derivative thereof, is radiolabeled with ¹²³I, ¹²⁴I, ¹²⁵I, ^{99m}Tc, ¹⁸F or ^{117m}Sn. In some examples, DaT2020, or a derivative thereof, is radiolabeled with ¹²³I, ¹²⁵I, ^{99m}Tc, or ^{117m}Sn, and counts are acquired by SPECT. In some examples, DaT2020, or a derivative thereof, is radiolabeled with ¹⁸F, ¹²⁴I, or ¹¹C, and the counts are acquired by PET. In some examples, about 0.25 mCi to about 10 mCi ¹²³I-labeled DaT2020, or a derivative thereof, is administered to the subject. In some examples, about 3 mCi to about 5 mCi ¹²³I-labeled DaT2020, or a derivative thereof, is administered to the subject. In some examples, the derivatives of DaT2020 comprise 2β-carbomethoxy-3β-(4-iodophenyl)tropane beta-CIT); 2β-carbomethoxy-3β-(4-iodophenyl)-N-(3-fluoropropyl)nortropane (FP-CIT) and TRODAT-1. In some examples, the ROI is the striatum and the subject not manifesting a clinical symptom is afflicted with Parkinson's disease, Lewy Body dementia, or diabetes.

The present disclosure provides a method of determining if a patient manifesting active tremor symptoms is afflicted with a non-dopaminergic movement disorder or a dopaminergic movement disorder, the method comprising: administering radiolabeled DaT2020, or a radiolabeled derivative thereof, to the patient; acquiring counts from the radiolabeled DaT2020, or derivative thereof, bound to DAT in the striatum of the patient, initiation of the acquisition of counts beginning at about 15 min after administration; measuring a number, density, and/or pattern of counts acquired; and comparing the number, density, and/or pattern of counts acquired from the striatum of the patient with the number, density, and/or pattern of counts obtained from an unafflicted, age-matched control subject that does not exhibit active tremor symptoms, the patient being afflicted with a dopaminergic movement disorder if the number, density and/or pattern of counts detected in the striatum of the patient is reduced relative to the counts, density, and/or pattern of counts obtained from the striatum of the unafflicted, age-match control subject, and the patient. being afflicted with a non-dopaminergic movement disorder if the number, density and/or pattern of counts detected in the striatum of the patient is not reduced relative to the counts, density, and/or pattern of counts obtained from the striatum of the unafflicted, age-match control subject.

In some examples, the non-dopaminergic disorder afflicting the patient manifesting active tremor symptoms is essential tremor. In some examples, the dopaminergic disorder afflicting the patient manifesting active tremor symptoms is Parkinson's disease, or Lewy Body dementia.

The present diagnostic and imaging methods aid in the differential diagnosis, leading to appropriate treatment of conditions where the functioning or dysfunctioning of DAT is a biomarker. These methods can also be used in clinical trials designed to evaluate the efficacy of new treatments for DAT dysfunction to stratify subjects according to disease stage. These methods are also useful for monitoring the effectiveness of treatments for and progression of DAT dysfunction over time.

Dysfunctions of DAT resulting in dopaminergic disorders are known in the brain and CNS, as well as outside of the CNS, including pancreas, kidney, and cardiovascular system.

In some cases, prevention of thyroid uptake of the iodine isotope is warranted. This can be accomplished by orally administering to the subject a Lugol solution, potassium iodide solution, or potassium perchlorate solution. In some cases, other preliminary steps are performed before the administration of radiolabeled DaT2020. For example, for SPECT detection and imaging of DAT binding, steps that counter serotonin-reuptake inhibitors, amphetamines, and sympathomimetics can be employed. The discontinuance of any medications that might interfere with the binding of DaT2020 to DAT may also be required. Such potentially interfering molecules include selective serotonin reuptake inhibitors and CNS stimulants. The thyroid blocker is administered per label instructions to ensure effective blocking before the scheduled administration of the agent.

To determine if a patient is afflicted with a non-dopaminergic or dopaminergic disorder, the patient is injected with a diagnostically effective amount of radiolabeled DaT2020.

Before agent administration, the subject is positioned in a SPECT or PET camera. Alternatively, sensors are placed adjacent to the ROI on the body which can collect radioactivity and which interfaces with a reader that communicates data to a computer.

SPECT and PET are procedures in which the isotope bound to DaT2020 or derivatives thereof is measured once the agent has been administered and has reached stable binding in the ROI. Once entering the blood stream, it travels throughout the body (e.g., brain, liver, kidney, heart, lungs, and the peripheral vascular system) and binds to DAT in various regions of the body including the striatum. The time it takes to reach stable binding (to eliminate background noise to form a clearer image) depends on the ROI and the depth of the tissue penetrated by the agent.

The camera captures energy produced by the radioactive decay of the radiolabeled DaT2020 or derivatives thereof (“tracer”). The radiolabel used for SPECT imaging emits energy in the form of individual photons or gamma rays, while the radiolabel used for PET imaging emits energy in the form of positrons. These positrons almost immediately collide with an electron and the energy produced becomes 2 photons emitted at roughly 180 degrees (2 photons moving in opposite directions).

In SPECT, the single emitted photon passes into the camera and strikes a scintillation crystal which, in turn, is viewed by a large number of photomultiplier tubes. The output voltages generated by the photomultiplier tubes are fed to a position circuit which produces four output signals. These signals contain information about the position and intensity of where the photon struck the crystal (scintillations). These signals (which “unblank” or “light up” the receiver, e.g., a cathode ray oscilloscope) for each photon are then fed into the memory circuitry of a computer and stored. The storage of these signals (the position and intensity of each photon becomes a count) allows their recall for digital processing from distinct time points or periods as well as the distinguishing counts from the components that make up the ROI that was scanned. Therefore, one can obtain counts and process them for quantitation at any single moment or over varying periods of time during a single scan session. In order to construct an image, a scan session must collect millions of counts over time in order to produce enough detail for a human reader to evaluate.

In PET, the collector mechanism and scintillation crystal are able to obtain position and intensity information from both photons and feed the information into the memory circuitry of a computer for storage and for later retrieval and processing.

Collection of counts is initiated at about 10 to 15 min after administration of the agent and is carried out for about 1 min to about 15 min.

To obtain an image, counts are collected beginning 15 min after administration of the radiolabeled agent and collection continues for 30 min (or 15-45 min after administration).

SPECT acquisition is performed on a SPECT/CT or stand-alone SPECT with at least two imaging heads fitted with collimators (parallel-beam and fan beam collimators with manufacturer specified (or measured according to NEMA standards) planar system resolution of <8 mm FWHM (in ‘air’ at 10 cm distance). Raw projection data (or counts) are acquired as described in Djang et al. (2013) Nuclear Med. Mol. Imaging 47(2):73-80), step-and-shoot mode with angle increments of 3° can be used. Alternatively, continuous rotation may be used. Full 3600 coverage of the area surrounding the ROI (e.g., when ROI is the striatum, the “area surrounding the ROI” would be the head) is required (i.e., 180° for each head of a dual-head camera). The number of sec per position depends on the sensitivity of the system, e.g., 30 sec to 40 sec.

The photopeak of the camera is set at 159 keV±10% and a 128×128 matrix is used. Optimal images are obtained when matrix size and zoom factors give a pixel size of 3.5 mm to 4.5 mm. Slices are about one pixel thick.

As described in Djang et al., 2012, ibid., review of projection data in cine mode and sinograms is performed for an initial determination of scan quality, patient motion, and artifacts. Motion correction algorithms may be used before reconstruction for minor movements, but rescanning is necessary if there is substantial head motion.

Iterative reconstruction (ordered subset expectation maximization [OSEM]) can be used, but filtered back-projection may be used. The reconstructed pixel size is 3.5 mm to 4.5 mm with slices one pixel thick.

Attenuation correction is done using an attenuation map measured from a simultaneously or sequentially acquired transmission or CT scan, or can be calculated, as with a correction matrix (see, Maebatake el al. (2015) J. Nucl. Med. Technol. 43: 41-46. doi:10.2967/jnmt.114.149401). The broad-beam attenuation coefficient is about 0.11 cm. Accuracy may be verified with an appropriate ¹²³I phantom (American College of Radiology (ACR). (2016). Site Scanning Instructions for the ACR Nuclear Medicine Phantom. In (pp. 18). Reston, Va.: American College of Radiology).

A low-pass filter (e.g., Butterworth) (Akahoshi et al. (2017) Medicine 96(45), e8484. doi:10.1097/md.0000000000008484) is useful. The filter preserves the linearity of the count rate response. Filtering includes either a 2-dimensional pre-filtering of the projection data or a 3-dimensional post-filtering of the reconstructed data.

Images are reformatted into slices in at least three planes depending on the ROI (axial, coronal, and sagittal). Transverse slices are parallel to a standard and reproducible anatomic orientation, such as the anterior commissure-posterior commissure line as used for brain MRI. This can be approximated by orientating the brain such that the inferior surface of the frontal lobe is level with the inferior surface of the occipital lobe. The canthomeatal plane, as routinely used for CT, is also acceptable. Activity in the striatum and the parotid glands, and the contours of the brain and the head, can usually be seen and can be used to assist realignment. A simultaneously acquired CT scan may allow precise realignment of the head.

Interobserver (expert image reader) variability is reduced by rigorously standardizing realignment and using predefined ROIs that are at least twice the full width at half maximum. Typically, this results in a smallest ROI dimension of 5 pixels to 7 pixels. In addition, three consecutive slices in the target region are used—those with the highest activity. Within the same center, the number of slices chosen are kept consistent.

After tracer administration, (with a tracer radiolabeled for scanning by PET, e.g., F-18), ‘PET scans are acquired for 10 min with the patient's eyes open in a dimly lit room with minimal auditory stimulation. Imaging acquisition is performed using a high-resolution PET-CT scanner (Gemini TF, Philips Medical Systems, Cleveland, Ohio) from the skull vertex to the base. PET scanner generates 90 contiguous transverse slices with an intrinsic resolution of 4.4 mm full-width half-maximum (FWHM) in all directions and an axial field of view of 18 cm. Attenuation correction is performed using a low-dose CT scan, 16-slice multidetector helical CT unit using the following parameters: 120 kVp; 30 mA; 0.5-s rotation time; 1.5-mm slice collimation, 2-mm scan reconstruction, with a reconstruction index of 2 mm; 60-cm field of view; 512×512 matrix.

Data acquired for imaging are reconstructed iteratively using a three-dimensional row action maximum-likelihood algorithm (RAMLA), often with low-dose CT datasets for attenuation correction in 3D mode

Image processing and calculation can be performed using Statistical Parametric Mapping 2 software (SPM2, Wellcome Department of Imaging Neuroscience, University College of London, UK) in conjunction with MATLAB version 7.0 (MathWorks Inc., Natick, Mass.) and FIRE (Functional Image Registration, Seoul National University, Seoul, Korea) program [24]. Image datasets of CTI format can be converted to ANALYZE format using the software MRIcro (www.mricro.com, Rorden and Brett, Columbia, S.C.).

The counts can be acquired until binding in the ROI is stable, and the method further comprises compiling an image of radiolabeled DaT2020, or a derivative thereof, bound to DAT in the ROI. For differential diagnosis of movement disorders, the ROI is the striatum of the patient. The image of the two halves is symmetrical if the patient is afflicted with a non-dopaminergic movement disorder, and the image is asymmetrical if the patient is afflicted with a dopaminergic disorder. The image is compiled from counts acquired by PET. Alternatively, the image is compiled from counts acquired by SPECT.

Alternatively, the pattern, level, and/or intensity of radiolabeled DaT2020 binding to DAT can be determined by the data captured by the sensors and sent, e.g., to a data reader attached to a computer. This method can be used for monitoring dopaminergic disorders affecting the brain or other ROI outside the brain.

The camera scans the patient to capture energy in the form of counts from radioactive decay of the radiolabeled DaT2020 or derivatives thereof (“tracer”). This scan does not need to capture the total number of counts needed to construct a clear image of the ROT, “counts”. During the scan, counts are continuously collected by the camera at preset coordinates focusing on the organ of interest and then digitized, stored, and then processed in near real time. The scan continues until a predefined condition is met.

One example of the condition is a predetermined threshold reached. This threshold may be determined by the number or density of counts (a SCORE) that a person without a dopaminergic disorder would be expected to have as demonstrated by comparison to a database of counts from these persons (or to a striatal phantom). If this condition is met, (evidence there is normal DAT density) there is an indication that the patient does not have a dopaminergic disorder. Once this condition is met, the processor analyzing the counts may output a signal to stop the camera.

Another example of the condition is the determination that the amount of energy being captured has stabilized. This condition may be met when the number of counts quantified by the processor is similar over a predetermined time period (e.g., every 30 sec). In the case of radiolabeled DaT2020, the energy stabilizes typically between 14 to 18 min. If the energy stabilizes, and the above first condition (e.g., the number, threshold, range, of counts) is not met, then this is an indication that the patient may have a dopaminergic disorder. In such instance, the processor outputs a signal to continue scanning the patient in order to collect enough data to create an image. The captured data is then processed by computer algorithms (computed tomography) to create a visual image representation of the ROI showing bright white where the Tracer has bound to DAT against a dark background.

The signals output by the processor (e.g., to stop the camera or to continue scanning the patient in order to collect enough data to create an image) may be a signal automatically controlling operation of the camera, or it may be a signal (e.g., an audio, visual, or tactile signal) to the operator of the camera.

Semiquantification is defined as the ratio of activity in a structure of interest (ROI) to activity in a reference region (Djang et al. (2012) ibid).

To date, the method used to limit human error in visually assessing images is to calculate a ratio of the counts from the ROI:counts from an area near the ROI that naturally has fewer dopamine transporters. It is this ratio [ROI-background/background] that provides a measure that is comparable regardless of tracer or camera used.

For example, if the goal is to determine if a movement disorder is parkinsonian (loss of DAT density) or non-parkinsonian the specific binding ratio (SBR, also referred to as the striatal binding ratio). SBRs are calculated after an image has been created by isolating (manually or through automation) the activity in the striatum and comparing it with activity in a low DAT density background area using the following formula:

$$Striatal binding ratio ( SBR ) = \frac{\begin{matrix}mean counts of striatal ROI - \\ mean counts of background ROI\end{matrix}}{mean counts of background ROI}$$

For both manual and automated semiquantification, SBRs for the left and right striatum are quantified separately, and the caudate and putamen are quantified separately; known anatomic lesions may influence the location of the striatal or background ROIs.

Techniques roughly fall into four categories: classic manual ROIs, manual volumes of interest (VOIs), more advanced automated systems using VOIs, and voxel-based mathematic systems (DatQUANT (Ge Healthcare, Little Chalfont, UK)

The classic and most widely used method applies ROI templates manually to one or more slices with the highest striatal activity. Manual VOI strategies stress accurate characterization of the putamen as the most sensitive region for distinguishing normal findings from Parkinsonian syndromes. For sampling the putamen, a small VOI not encompassing the whole structure may be considered. Mid-putaminal VOIs provide accurate manual results. Automated VOI systems incorporating the whole striatum using individualized VOIs, either based on the ¹²³I-labeled tracer SPECT data or on a coregistered anatomic scan, produce more objective, observer-independent results and are faster although not widespread.

The following method improves on prior analysis methods because it is performed significantly faster as it does not require a full image to be generated prior to analysis. It also uses the patient as their own control. Software (DaTsnap) captures raw counts and calculates a caudate to putamen ratio (CPR) ratio of activity on the side of the brain contralateral to the movement disorder symptoms. The threshold for stopping the scan is when this ratio exceeds the normal ratio value.

$$CPR ( Caudate to putamen ratio ) = \frac{Mean count of caudate ROI}{Mean count of putamen ROI}$$

When this threshold is exceeded before the full imaging time has elapsed, the patient is afflicted with a dopaminergic movement disorder, the ratio of the patient's caudate to putamen will be higher relative to the normal ratio threshold. If the patient is afflicted with a non-dopaminergic movement disorder, the ratio of mean counts acquired from the patient's caudate to putamen will be the same, or similar to, the ratio obtained from an unafflicted subject.

To get an accurate determination of the level or amount of DAT in, e.g., the striatum or other ROI, injection of radiolabeled DaT2020 should be monitored starting at the injection to make sure it has entered the circulating system from its injection site. This can be accomplished by any method known in the art, such as, but not limited to, the LaraSystem (Lucerno Dynamics).

For example, should the administered radiolabeled DaT2020 cause an infiltration at the injection site or should it be limited to the site of a venous blockage, the label will not, or more slowly, get to the brain or ROI, or very little of it will get to the site of DATs, resulting in a false reading or no or low DAT, and potentially leading to a false diagnosis of a dopaminergic disorder.

The present disclosure provides a method of determining if a subject not manifesting a clinical symptom of a dopaminergic disorder is afflicted with the dopaminergic disorder, comprising: administering a first radiotracer; waiting for a first time interval; administering a second radiotracer; waiting for a second time interval; acquiring counts from the first radiotracer and the second radiotracer, bound to DAT in a region of interest (ROI) of the body of the subject; measuring a number, density, and/or pattern of counts acquired; and comparing the number, density, and/or pattern of counts acquired from the ROI of the subject with the number, density, and/or pattern of counts obtained from an unafflicted, age-matched control subject, the patient being afflicted with a dopaminergic movement disorder if the number, density and/or pattern of counts detected in the ROI is reduced relative to the counts, density, and/or pattern of counts obtained from the ROI the unafflicted, age-match control subject, wherein the first radiotracer and the second radiotracer have different binding affinities for the dopamine transporter (DAT) and different pharmacokinetics.

The present disclosure provides a method of determining if a subject not manifesting a clinical symptom of a dopaminergic disorder is afflicted with the dopaminergic disorder, comprising: administering a second radiotracer; waiting for a first time interval; administering a first radiotracer; waiting for a second time interval; acquiring counts from the first radiotracer and the second radiotracer, bound to DAT in a region of interest (ROI) of the body of the subject; measuring a number, density, and/or pattern of counts acquired; and comparing the number, density, and/or pattern of counts acquired from the ROI of the subject with the number, density, and/or pattern of counts obtained from an unafflicted, age-matched control subject, the patient being afflicted with a dopaminergic movement disorder if the number, density and/or pattern of counts detected in the ROI is reduced relative to the counts, density, and/or pattern of counts obtained from the ROI the unafflicted, age-match control subject, wherein the first radiotracer and the second radiotracer have different binding affinities for the dopamine transporter (DAT) and different pharmacokinetics.

In some examples of the methods disclosed herein, the first time interval is between about 5 to about 30 minutes, between about 0.5 to about 1 hour, between about 1 to about 2 hours, between about 2 to about 3 hours, between about 3 to about 4 hours, between about 4 to about 5 hours, or between about 5 to about 6 hours. In some examples of the methods disclosed herein, the second time interval is between about 5 to about 30 minutes, between about 0.5 to about 1 hour, between about 1 to about 2 hours, between about 2 to about 3 hours, between about 3 to about 4 hours, between about 4 to about 5 hours, or between about 5 to about 6 hours. In some examples of the methods disclosed herein, the binding affinity of the first radiotracer for DAT is between about 2- to about 50-fold greater than the binding affinity of the second radiotracer for DAT. In some examples of the methods disclosed herein, the pharmacokinetics of the first radiotracer is between about 2- to about 500-fold greater than the pharmacokinetics of the second radiotracer. In some examples of the methods disclosed herein, the first radiotracer is [¹²³I E-2β-carbomethoxy-3β-(4-fluorophenyl)-N-(3-iodo-E-allyl) nortropane (DaT2020). In some examples of the methods disclosed herein, the second radiotracer is N-ω-fluoropropyl-2β-carbomethoxy-3β-(4-iodophenyl) nortropane (DaTSCAN).

In some examples of the methods disclosed herein, the sequence of administration of the first radiotracer and second radiotracer is determined by disease, pharmacokinetics and binding affinity to DAT.

Reference will now be made to specific examples illustrating the disclosure. It is to be understood that the examples are provided to illustrate exemplary embodiments and that no limitation to the scope of the disclosure is intended thereby.

### EQUIVALENTS

Those skilled in the art will recognize, or be able to ascertain, using no more than routine experimentation, numerous equivalents to the specific embodiments described specifically herein.

1. A pharmaceutical packaging unit comprising:
a first radiotracer; and
a second radiotracer,
wherein the first radiotracer and the second radiotracer have different binding affinities for the dopamine transporter (DAT).

2. The pharmaceutical packaging agent of claim 1, wherein the binding affinity of the first radiotracer for DAT is between about 2- to about 50-fold greater than the binding affinity of the second radiotracer for DAT.

3. The pharmaceutical packaging agent of claim 1, wherein first radiotracer comprises [¹²⁵I E-2β-carbomethoxy-3β-(4-fluorophenyl)-N-(3-iodo-E-allyl) nortropane (DaT2020).

4. The pharmaceutical packaging agent of claim 1, wherein the second radiotracer comprises N-ω-fluoropropyl-2β-carbomethoxy-3β-(4-iodophenyl) nortropane (DaTSCAN).

3. A pharmaceutical packaging unit, comprising:
a first radiotracer; and
a second radiotracer,
wherein the first radiotracer and the second radiotracer have different pharmacokinetics.

4. The pharmaceutical packaging unit of claim 3, wherein the pharmacokinetics of the first radiotracer is between about 2-fold to about 500-fold greater than the pharmacokinetics of the second radiotracer.

5. The pharmaceutical packaging agent of claim 3, wherein first radiotracer comprises [¹²³I E-2β-carbomethoxy-3β-(4-fluorophenyl)-N-(3-iodo-E-allyl) nortropane (DaT2020).

6. The pharmaceutical packaging agent of claim 3, wherein the second radiotracer comprises N-ω-fluoropropyl-2β-carbomethoxy-3β-(4-iodophenyl) nortropane (DaTSCAN).

7. A pharmaceutical packaging unit comprising:
a first radiotracer; and
a second radiotracer,
wherein the first radiotracer and the second radiotracer have different binding affinities for the dopamine transporter (DAT) and different pharmacokinetics.

8. The pharmaceutical packaging of claim 7, wherein the binding affinity for DAT of the first radiotracer is between about 2-fold to about 500-fold greater than the binding affinity for DAT of the second radiotracer, and the pharmacokinetics of the first radiotracer is about 2-fold to about 500-fold greater than the pharmacokinetics of the second radiotracer.

9. The pharmaceutical packaging agent of claim 7, wherein first radiotracer comprises [¹²³I E-2β-carbomethoxy-3β-(4-fluorophenyl)-N-(3-iodo-E-allyl) nortropane (DaT2020).

10. The pharmaceutical packaging agent of claim 7, wherein the second radiotracer comprises N-ω-fluoropropyl-2β-carbomethoxy-3β-(4-iodophenyl) nortropane (DaTSCAN).

11. A method of determining if a subject not manifesting a clinical symptom of a dopaminergic disorder is afflicted with the dopaminergic disorder, comprising:
administering a first radiotracer; waiting for a first time interval;
administering a second radiotracer; waiting for a second time interval,
the first radiotracer and the second radiotracer having different binding affinities for the dopamine transporter (DAT) and different pharmacokinetics;
acquiring counts from the first radiotracer and the second radiotracer, bound to DAT in a region of interest (ROI) of the body of the subject;
measuring a number, density, and/or pattern of counts acquired; and
comparing the number, density, and/or pattern of counts acquired from the ROI of the subject with the number, density, and/or pattern of counts obtained from an unafflicted, age-matched control subject,
the patient being afflicted with a dopaminergic movement disorder if the number, density and/or pattern of counts detected in the ROI is reduced relative to the counts, density, and/or pattern of counts obtained from the ROI the unafflicted, age-match control subject.

12. The method of claim 11, wherein the first time interval is between about 5 minutes to about 6 hours.

13. The method of claim 11, wherein the second time interval is between about 5 minutes to about 6 hours.

14. The method of claim 12, wherein the second time interval is between about 5 minutes to about 6 hours.

15. The method of claim 11, wherein the binding affinity of the first radiotracer for DAT is between about 2-fold to about 500-fold greater than the binding affinity of the second radiotracer for DAT.

16. The method of claim 11, wherein the first radiotracer comprises [¹²³I E-2β-carbomethoxy-3β-(4-fluorophenyl)-N-(3-iodo-E-allyl) nortropane (DaT2020).

17. The method of claim 11, wherein the second radiotracer comprises N-ω-fluoropropyl-2β-carbomethoxy-3β-(4-iodophenyl) nortropane (DaTSCAN).

18. A method of determining if a subject not manifesting a clinical symptom of a dopaminergic disorder is afflicted with the dopaminergic disorder, comprising:
administering a second radiotracer; waiting for a first time interval;
administering a first radiotracer; waiting for a second time interval,
the first radiotracer and the second radiotracer have different binding affinities for dopamine transporter (DAT) and different pharmacokinetics;
acquiring counts from the first radiotracer and the second radiotracer, bound to DAT in a region of interest (ROI) of the body of the subject;
measuring a number, density, and/or pattern of counts acquired;
and comparing the number, density, and/or pattern of counts acquired from the ROI of the subject with the number, density, and/or pattern of counts obtained from an unafflicted, age-matched control subject,
the patient being afflicted with a dopaminergic movement disorder if the number, density and/or pattern of counts detected in the ROI is reduced relative to the counts, density, and/or pattern of counts obtained from the ROI the unafflicted, age-match control subject.

19. The method of claim 18, wherein the binding affinity of the first radiotracer for DAT is between about 2-fold to about 500-fold greater than the binding affinity of the second radiotracer for DAT.

20. The method of claim 18, wherein the first radiotracer comprises [¹²³I E-2β-carbomethoxy-3β-(4-fluorophenyl)-N-(3-iodo-E-allyl) nortropane (DaT2020).

21. The method of claim 11, wherein the second radiotracer comprises N-n)-fluoropropyl-2β-carbomethoxy-3β-(4-iodophenyl) nortropane (DaTSCAN).
